(19) United States
(12) Patent Application Publication
(10) Pub. No.: US 2009/0220940 A1
(43) Pub. Date: Sep. 3, 2009

(54) Method for Testing the Integrity of Membranes

(76) Inventors: Ovadia Lev, Jerusalem (IL); Genia Gun, Jerusalem (IL); Vitaly Gitis, Arad (IL)

Correspondence Address: NIXON & VANDERHYE, PC, 901 NORTH GLEBE ROAD, 11TH FLOOR, ARLINGTON, VA, 22203, US

(21) Appl. No.: 12/083,600

(22) PCT Filed: Oct. 17, 2006
(86) PCT No.: PCT/IL2006/001196

§ 371 Date: Apr. 29, 2009

Related U.S. Application Data
(60) Provisional application No. 60/726,623, filed on Oct. 17, 2005.

(51) Int. Cl. C12Q 1/70; C12N 7/00

(52) U.S. Cl. 435/5; 435/235.1

(57) ABSTRACT

A method for evaluating the integrity of microfiltration, ultrafiltration and nanofiltration membranes, which method comprises passing a liquid that contains a substantially mono-dispersed population of nano-probes through said membrane to form a permeate and testing said permeate for the presence of said nano-probes, wherein the non-detection of said nano-probes in said permeate indicates that said membrane is substantially intact.

### FIELD OF THE INVENTION

The present invention concerns methods for evaluating the integrity of filtration membranes by the use of probes.

### BACKGROUND OF THE INVENTION

Membrane systems are being increasingly used for the direct treatment of drinking water. However, the lack of means to accurately monitor membrane integrity over long-term operation is preventing the full exploitation of the potential of ultrafiltration (UF), microfiltration and nanofiltration technologies for water treatment. Membrane integrity, i.e., the absence of feed leakage through affected or broken membranes, or passage through seals, can be compromised as a result of factory defects, improper shipping and maintenance, faulty integrity tests, excessive exposure to chemicals during chemical clean-up or natural wear under continuous operation.

Several direct and indirect integrity tests for detection of system breaches are currently in use.

The direct integrity testing methods include the acoustic noise analysis, the bubble point test, the pressure decay test (PDT), and the diffusive air flow (DAF) test. However, it should be noted the minimal detectable level for the aforementioned pressure-integrity tests (i.e., bubble point test, PDT and DAF test) is 2-3 μm, a detection limit that is sufficient to assure compliance with removal requirements for Giardia (˜6-20 μm), Cryptosporidium (˜4-6 μm) and other parasites but not viruses (<<0.1 μm). Furthermore, the PDT and the DAF tests may at times cause membrane breakage during the test and may yield false-positive results due to the membrane being partially unwetted (after backwash) or due to seasonal viscosity changes (EPA, Membrane Filtration Guidance Manual, U.S. Environmental Protection Agency Office of Water (4601), EPA 815-d-03-008, June 2003). These tests also suffer from subjective interpretation of pressure deterioration. Thus, plants that rely on pressure integrity tests alone are likely to continue their operation beyond the point where their membrane integrity has been compromised.

Indirect integrity tests include particle counting, turbidity monitoring and surrogate challenge tests. In all of these tests, changes in filtrate quality are monitored by comparing test probe levels with a previously established baseline level. Recently, van Hoof et al. (Development of a new integrity testing system, Proceedings of the IWA/AWWA Conference Membranes in Drinking and Industrial Water Production, Mulheim an der Ruhr, Germany, September 2002) proposed an on-line surrogate challenge test using a spike injection or a constant feed of powdered activated carbon (PAC), followed by optical monitoring of filtrate quality with particle or turbidity monitors.

EP 1647345 describes an integrity test suitable for virus removal membrane, wherein a colloidal solution which contains gold particles is filtered through the membrane and the absorption spectrum of the feed stream and the filtrate are measured in order to assess the integrity of the membrane. The authors show that up to 2.5 log removal (see definition hereinbelow) can be detected by this method.

Gitis V., Adin A., Nasser A., Gun J. and Lev O. [Water research 36, p. 4227-4234 (2002)] described labeled bacteriophages for studying the pollutant transport in various environmental processes.

### SUMMARY OF THE INVENTION

It has now been found that it is possible to accurately and consistently evaluate the integrity of a membrane by passing therethrough a population of nano-probes characterized by a high degree of monodispersivity and enhanced observability, collecting the permeate (i.e., the liquid that passes through the membrane) and testing the same for the presence of said nano-probes.

The term “a population of nano-probes characterized by a high degree of monodispersivity” and the like, as used herein, relates to a population of chemical or biological species whose average diameter is in the range of between 1 nm and 500 nm, and more preferably in the range of between 1 and 100 nm, wherein said population is characterized by a narrow size distribution, such that not more than 0.5% of the particles of said population, and preferably not more than 0.01% thereof have a particle size which is smaller than half of the average size of the nano-probe particles.

Another property that is met by the nano-probes of the present invention, in addition to the characteristic particle size distribution as defined above, is that they are readily detectable. It has been found by the inventors that a substantially mono-dispersed population of the nano-probes (which usually can be observed only at relatively high concentration) can become observable by means of coupling thereto, either chemically (covalently) or physically, a reporting molecule, namely, a molecular probe that is preferably optically detectable (such as a dye compound or a fluorescent dye compound) or can participate in a chemical reaction which involves the formation of one or more optically detectable forms, as will be discussed in more detail below.

Accordingly, in a first aspect, the present invention provides a method for evaluating the integrity of a microfiltration, ultrafiltration or nanofiltration membranes, which method comprises passing through said membrane a liquid that contains a substantially mono-dispersed population of nano-probes to form a permeate and testing said permeate for the presence of said nano-probes, wherein the non-detection of said nano-probes in said permeate indicates that said membrane is substantially intact and/or is suitable for intended purpose (e.g., pathogen removal, water purification retention, ultrafiltration, etc.)

Methods for determining the particle size distribution of a given population of nano-probes and confirming its uniformity, if required, are well known in the art and include electron microscopy (e.g., TEM) and dynamic laser diffraction (e.g., Malvern).

Membranes whose integrity may be evaluated according to the present invention include ultrafiltration, microfiltration and nanofiltration membranes in the form of sheets, plates, fibers or any other configuration. Microfiltration membranes may contain pores with size distribution between 100 nm to more than 1 micron, ultrafiltration membranes contain pores of about 10-100 nm and nanofiltration membranes have pore size distribution between 1 to 10 nm.

Nano-probes which may be suitably used according to the present invention include biological entities, which in the context of the present invention relate specifically to bacteria, viruses (including bacteriophages), proteins and nucleic acids or entities containing the same. It may be appreciated that the viruses/bacteriphages of the same strain, proteins having the same sequence and nucleic acids having the same sequence are all of uniform size and thus comply with the definition “substantially mono-dispersed population”. However, the term “biological entity” should not be limited to the aforementioned examples.

Accordingly, microorganisms including viruses and bacteriophages and protein biomolecules at large are inherently characterized by monodispersivity and are therefore especially useful as nanoprobes in accordance with the present invention. Viruses (and bacteriophages) may be obtained from various commercial sources such as DSMZ-Deutsche Sammlung von Mikroorganismen und Zellkulturen GmbH, Braunschweig, Germany; ATCC—American Type Culture Collection and NCTC—The National Collection Of Type Cultures, UK. Suitable examples of viruses that may be used in connection with the present invention are depicted in Table 1:

TABLE 1
The main characteristics of bacteriophages

| | | Particle | | | Isoelectric |
|---|---|---|---|---|---|
| Virus | Family/group | morphology | Nucleic acid^{a} | Size (nm) | point, pH |
| MS2 | Leviviridae | Symmetry | Linear ss RNA | 26-27 | 3.9 |
| | | icosahedral | | | |
| T4 | Myoviridae | Tailed phage | Linear ds DNA | Head 65 × 80, | 3.2 |
| | | | | tail 120 × 20 | |
| Phi X174 | Microviridae | Icosahedral | Circular ss DNA | 31-32 | 6.6 |
| Phi 6 | Cystoviridae | Isometric | Linear ds RNA | 22 | 5.3 |
| PRD1 | Tectiviridae | Symmetry | Linear ds DNA | 65 | 4.2 |
| | | icosahedral | | | |

^{a}ss = single-strained; ds = double-strained

As pointed out hereinabove, an important property that is satisfied by nano-probes to be used according to the present invention is their enhanced observability. In order to facilitate a rapid quantification of the nano-probes in the permeate generated by the membrane and thus allow the detection of breaches in membranes under field conditions within a very short time, it is preferred to use nano-probes that can be readily observed in very low concentration.

Thus, according to a particularly preferred embodiment of the invention, the nano-probe is coupled to a reporting molecule, wherein said reporting molecule is either an optically or electrochemically detectable compound, or said reporting molecule is capable of participating in a chemical transformation or in a physical process which involve the formation of one or more intermediates and/or products that are optically or electrochemically detectable.

More specifically, the viruses (including bacteriophage—a class of viruses that affect bacteria) may be suitably labeled with a molecule that can be directly observable. The reporting molecule (hereinafter sometimes referred to as a molecular probe) may be directly detected by optical, electrochemical, piezoelectric, immunological or biological (binding) means. Examples of optical detection may be by using a fluorescent or other optically detectable compound (optically observable dye molecules). For example fluorescent or other detectable probe (e.g., fluorescein-5-isothiocyanate, fluorescein, 5-(4,6-dichlorotriazinyl)aminofluorescein and rhodamine B) may be coupled to the virus by means of known techniques, such as those described by Banks et al. [Bioconjugate Chem. 6, 447-458 (1995)] and Gitis et al. (supra). Briefly, a buffer containing the bacteriophages may be mixed with the fluorescent probe optionally in the presence of a co-solvent and/or a coupling agent, preferably under stirring for several hours. The resulting labeled bacteriophage mixture may be subsequently purified by means of dialysis, gradient centrifugation, centrifugation or another separation technique.

Alternatively, the virus/bacteriophage is coupled to a molecular probe which is capable of participating in a chemical reaction (either as a reactant or as a catalyst), which reaction results in the formation one or more intermediates/products having improved optical detectability. The conjugation of the molecular probe to the virus/bacteriophage may be carried out either directly (covalently) or through a spacer molecule by methods known in the art. According to a preferred embodiment, the reporting molecule/molecular probe that is coupled to the virus/bacteriophage is an enzyme that catalyzes the oxidation of a substrate (in the presence of an oxidation agent) to form an optically detectable oxidation product. Preferably, the catalyzed reaction produces two or more optically or electrochemically detectable product molecules per one molecule of substrate. For example, the enzyme horse radish peroxidase can catalyze the oxidation of pyrogalol to purpurogallin (in the presence of H₂O₂), and the latter can be readily observed by spectrophotometric detection. The signal of the purpurogallin is characterized by an absorbance wave starting at a wavelength of approximately 530 nm with a shoulder at 420 nm. The absorbance at 420 nm can be used for quantification of the purpurogallin and thereby for characterization of the enzymatic activity and thereby the number of the labeled phages in the sample. Knowledge of the number of enzyme labels per phage is not necessary since the LRV (log removal) is a relative quantity (between input and output). It is important to carry out the spectrophotometric quantification after a constant time interval from the incorporation of the pyrogalol into the HRP containing solution since the production of purpurogallin is commulative. Typical reaction time is in the range 20 sec to 30 minutes. The temperature is held constant (preferably at 25° C.), wherein the pH is around pH 6 and should be maintained constant for the measurement of the initial solution and the permeate. It has been observed that the conjugation of horse radish peroxidase bacteriophages does not interfere with the relevant properties of said bacteriophages, and in particular the monodispersity of the nano-probes is retained.

Accordingly, in another aspect, the present invention provides the following nano-probes, which have been found to be particularly preferred in performing the method of the invention:

A bacteriophage conjugated to a catalytic entity that is capable of catalyzing a reaction that produces two or more optically or electrochemically detectable products per one molecule of substrate. Preferably, the catalytic entity which is appended to the bacteriophage changes the size of the virus by not more than 25% relative to its original size, and wherein the sign of the zeta potential of the original phage is not altered by the conjugation of said catalytic entity. Preferably, the catalytic entity conjugated to the bacteriophage is a peroxidase or glucose oxidase enzyme.

The invention also provides inactivated virus conjugated to a catalytic entity, wherein said catalytic entity is capable of catalyzing a reaction that produces two or more optically or electrochemically detectable products per one molecule of substrate.

The invention also provides a virus conjugated to an enzyme or DNAZYME entity, wherein said entity is capable of catalyzing a reaction that produces two or more optically or electrochemically detectable products per one molecule of substrate.

Proteins for use in the present invention may be purified from biological materials, expressed recombinantely and in some cases synthesized chemically. The possible proteins list includes, but is not limited to, hide glue, casein, albumin, lysozyme, myoglobin, hemoglobin, etc. A dye molecule, a fluorescent or other detectable probe may be coupled to the protein using the techniques described herein above [Banning N, Toze S, Mee B J (2002) Escherichia coli survival in, groundwater and effluent measured using a combination of propidium iodide and the green fluorescent protein. JOURNAL OF APPLIED MICROBIOLOGY 93 (1): 69-76].

Nucleic acids such as DNA and RNA for use in the present invention may be synthesized chemically, translated and/or transcribed from recombinant systems and/or purified from biological materials using techniques that are well known in the art.

If required, the biological material may be subjected to one or more purification procedures, for example centrifugation, gradient centrifugation, electrophoresis, gel permeation, size exclusion chromatography or membrane fractionation in order to improve the uniformity of the particle size distribution according to procedures well known in the art. [Harrison R. G., Todd P., Rudge S. R. and Petrides D. P. Bioseparations science and engineering, Oxford University Press, NY 2003).

In order to test the integrity of a membrane according to the present invention, a liquid containing the nano-probes is filtered through the membrane. The concentration range of nanoprobes in the feed is in the range of between 0.01% and 10% nm, and more preferably in the range of between 0.1% and 1%, the applied pressure is in the range of between 0.1 bar and 5 bars, and more preferable between 0.5 bar to 2 bars. The permeate generated by the membrane is qualitatively and/or quantitatively tested for the presence of the nano-probe. Preferably, however, in order to determine the degree of retention of the nano-probes by the tested membrane, the concentration of said nano-probes in the feed and in the permeate need to quantified. Most conveniently, the degree of retention is expressed by means of the Log Removal Value parameter:

$$LRV = {log}_{10} ( \frac{{C}_{f}}{{C}_{p}} ) \qquad ( 1 )$$

Where LRV=log removal value; C_{f}=concentration of the nano-probes in the feed and Cₚ=concentration of nano-probes in the permeate. It is important to note that Log Removal Values attainable by the use of the biological nano-probes described above are preferably above 3. In order to guarantee that the removal of pathogen of a specific size surpass a certain value it is important that majority of probe particles will be smaller than the target pathogen (or virus). However if there is a large fraction of probe particles which are too small (smaller than the membrane cutoff of the membrane) than the lack of removal or high observable LRV (of the probe particles) will not necessarily indicate on large LRV of the target pathogen. In order to minimize this inherent duality monodispersity of the probe particles is important. Additionally, the average probe particle should be as close as possible in size (and other physical parameters such as zeta potential and density) to the target pathogen. Moreover, the tailing of the size distribution histogram of the probe particles towards the small sizes should be minimal. Note that the use of bioentities and particularly viruses and globular proteins guarantees the last request by the nature of the bioentities. The bioentities can be aggregated (and give larger particles) but at normal conditions (pH 3-9) and without the presence of aggressive chemicals (such as surfactants and oxidizers) the fundamental unit size of the bioentities will remain constant without tailing to lower dimensions. Our procedure using bioentities and highly monodispersed particles is therefore most suitable for conditions where high LRV (preferably above 3) should be demonstrated.

As discussed in detail above, it is preferred to quantify the concentrations of the nano-probe in the feed and in the permeate using readily detected and/or measured physical property associated with said nano-probe. Preferably, such a property will be a form of electromagnetic radiation, such as visible light or ultraviolet radiation. For example, when the nano-probe is a fluorescent dye labeled biological entity as described above, the fluorescence spectra of the feed and permeate may be recorded using fluorescence spectrometer and the intensities measured in the two samples are compared against a calibration curve, whereby the concentration of the nano-probes in the samples are readily determined.

Other useful properties include electrochemical response (e.g. by conjugation of ferrocene, or hydroquinone moieties) or other forms of optical observability, For example, chemoluminescence or light absorption. These techniques are well known to analytical chemists and are described in the different chapters of the book principles of instrumental analysis Fifth edition, authored by D A Skoog, E J Holler and T A Nieman, Saunders College publishers, Philadelphia, 1998.

It should be noted that the integrity test provided by the present invention using the biological nano-probes may be applied on-site and also on-line. Thus, in another aspect, the present invention provides a method that is carried out on-line, wherein the liquid that contains the nano-probes is added to the feed of the membrane module and one or more sensors are used to detect said nano-probes in the permeate by measuring at least one detectable property associated with said nano-probes, for example by measuring an optical property such as the absorbance and or fluorescence spectra. As explained above, the optical property of the nano-probes may be directly measured in the filtrate or may require triggering a chemical reaction therein in the participation of a reporting molecule that is attached to the nano-probe, which chemical reaction is associated with the formation of an optically detectable compound.

In addition to nano-probes which are biological entities, as described above, nano-probes which are metal nanoparticles, ceramic probes or organic polymer beads (e.g. latex beads) may also be practiced according to the present invention. The aforementioned nano-probes may be prepared by methods known in the art (for example, the synthesis of metal particles is described herein below) and are ready for use following a clean-up procedure such as gradient centrifugation, size exclusion chromatography or electrophoretic separation in order to remove the “tail” of small dimension particles and arrive at particles population satisfying the requirements mentioned above. If desired, the chemical conjugation of the nanoparticle to a reporting molecule (having electrochemical or optical detectability) is subsequently carried out.

The inventors have also found that it is possible to accurately and consistently evaluate the integrity of a membrane by passing therethrough gold nanoparticles (preferably after appropriate clean up to remove a large portion of the smaller particles), collecting the permeate and analyzing the same for the presence of said gold nano-particles by means of one or more electrochemical techniques.

Accordingly, in another aspect, the present invention provides a method for evaluating the integrity of a membrane, which method comprises passing gold nanoparticles dispersed in an aqueous medium through said membrane to form a permeate, sampling the permeate, and electrochemically testing the sample for the presence of gold. More specifically, the electrochemical testing includes introducing an oxidizing agent into the sample to form gold ions in a solution, depositing metal ions present in the solution onto a working electrode and subsequently applying a progressively varied anodic potential to said working electrode capable of releasing gold therefrom, whereby the gold quantity is determined. The aforementioned stages will now be described in detail.

According to this aspect of the invention, the nano-probes are nanoparticles provided in the form of gold cores coated with one or more organic layers, said gold nanoparticles being dispersed within a suitable liquid, which is preferably an aqueous medium.

The organic coating provided on the surface of the gold cores prevents the agglomeration of the nanoparticles in the aqueous medium, thus maintaining the stability of the colloidal system, such that a substantially uniform particle size distribution of said nanoparticles population is retained during a long storage period and over broad concentration and/or pH ranges. To this end, the organic coating preferably comprises ionizable functional groups, and more specifically, carboxylic groups (—COOH), which, when present in their corresponding ionized form, produce electrical repulsion forces between the individual nanoparticles in the aqueous suspension. Alternatively, it is possible to use organic coating composed of bulky, long-chains molecules capable of causing steric repulsion between the nano-particles. To this end, sulfur-containing compounds, and especially alkanethiols may be used, in view their ability to adhere onto the gold surface by means of forming strong Au—S bonds. Possible alkane thiol compounds include propylthiol, butylthiol, pentylthiol and other straight chain alkyl thiols.

The preparation of the nanoparticles described hereinabove may be accomplished by methods known in the art, as described for example by Shipway et al. (Chem. Phys. Chem. 1 p. 18-52 (2000)]. More specifically, the preparation of gold nanoparticles may be briefly described as follows. A compound of gold, in which the gold is preferably present as Au³⁺, (e.g., hydrogen aurichloride, HAuCl₄) is dissolved in a suitable solvent, which is most preferably water, and is reacted with a reducing agent, following which the gold particles are formed. It is especially preferred to use an organic reducing agent, and more specifically citric acid or a salt thereof, since the formed gold particles are in-situ coated with one or more layers of the organic oxidation products of said reducing agent, thus guaranteeing the formation of a mono-dispersed population. It has been observed that the resulting colloidal system is sufficiently stable, as will be discussed in more detail below, and therefore does not necessarily require the introduction of a dispersant or surfactant thereto.

The oxidation-reduction reaction described above may be carried out at temperature in the range between 80 to 100° C. for about 5 min. The concentration of the gold in the reaction mixture is typically in the range between 5 and 500 ppm.

If desired, the citrate-based coating may be displaced by another organic coating by treating the citrate-stabilized nanoparticles prepared as described above with alternative organic compounds, e.g. alkanethiolate, alkaneamine, tripeptide, p-thiophenol, tiopronin, 2-(dimethylamino)ethanethiol, mercaptoethanesulfonate, etc.

In order to test the integrity of a membrane according to the present invention, the aqueous medium containing the gold nanaparticles dispersed therein is passed through the membrane and its presence in the permeate is determined electrochemically. Filtration parameters that may be employed are described hereinabove.

The gold that is potentially present in the permeate is in a metal form, and hence not available for stripping voltammetry. The metal is therefore subjected to an oxidation reaction:

Au⁰→Au³⁺

wherein the oxidizing agent is preferably a concentrated aqueous solution of a strong acid, e.g., hydrochloric acid or nitric acid, and most preferably, a combination thereof, as illustrated by the following table:

TABLE 2
Dissolution of gold nanoparticles in aqua regia.

Aqua regia concentration, M/Color, ABS

| Time | 0.5M HCl | 1M HCl | 2M HCl | 3M HCl |
|---|---|---|---|---|
| Immediately | 0.016 | 0.011 | 0.003 | ND |
| 1 hour | 0.009 | 0.011 | 0.003 | ND |
| 2 hours | 0.001 | ND | ND | ND |
| 3 hours | 0.001 | ND | ND | ND |

ND = below detection limit
Conditions: 1 M HNO₃; 50° C.

The oxidation reaction using the aforementioned combination of acids is carried out at a temperature in the range between 10° C. and 35° C., and may last for about 1 to 10 min. The completion of the oxidation reaction may be determined with the aid of spectrophotometry, with the disappearance of the 535 nm peak indicating the complete interconversion Au⁰→Au³⁺, i.e. from colloidal to ionic gold required for the subsequent application of anodic stripping voltammetry.

The resulting sample solution, which contains the gold ions, is now subjected out an anodic stripping voltammetry in a suitable electrochemical cell. In the stripping voltammetry procedure according to the present invention, gold ions present in a volume of the tested sample are first deposited on a working electrode in contact with said sample solution by applying a first (negative) potential on said working electrode, following which the potential on said working electrode is continuously or incrementally varied arriving at positive values whereby the metallic gold is oxidized and is stripped from said working electrode back into solution.

In its most general form, an electrochemical cell suitable for anodic stripping voltammetry comprises an enclosure for holding a volume of the samples solution, at least one working electrode, which may be made of graphite or glassy carbon, a counter electrode, which may be made of Pt and a reference electrode (e.g., Ag/AgCl). Stirring means are also provided. Suitable electrochemical cells for anodic stripping voltammetry are well known in the art.

One preferred set-up of an electrochemical device to be used according to the present invention is illustrated in FIG. 1. The sample holder 38 which holds the sample solution to be tested is preferably a flat plastic (acrylic) or glass cell, whose volume is in the range between 10 and 50 ml. A working electrode assembly 32 is centrally placed within the cell, wherein said assembly is provided in the form of a plurality of circularly arranged electrodes that are electrically connected by means of a conductive strip, e.g., a copper strip. The ratio of electrode surface area to the sample is preferably not less than 8 cm²/ml. For example, for a 5 ml volume of the tested sample, it is possible to use six disposable graphite electrodes (7 cm long, 0.7 mm I.D.) available from Sacura Mines, Japan. In addition, the device comprises commonly used counter electrode and reference electrode. The counter electrode 37 is most simply a platinum wire which may directly contact the solution or is being contained in a suitable glass compartment or shell. The reference electrode 34 is a preferably Ag/AgCl electrode which is connected to the sample through a salt bridge 33. A potentiostat-galvanostat is indicated by numeral 35 whereas the stirring means is indicated by numeral 31.

In operation, a volume (5-40 ml) of tested sample is placed in the sample holder and the working electrode assembly is initially connected in a cathodic mode to the power source. The cathodic potential is typically in the range of −0.4 V to −1.1 V, and more preferably at about −0.7 V. The electroplating of the electrode is preferably allowed to continue for 1 to 15 minutes. It has been found that the addition of mercury ions (Hg²⁺) at a concentration in the range of 50 to 200 ppm into the tested sample may considerably facilitate the analysis. The presence of mercury ions assist in the cathodic accumulation, allowing the deposition stage to be relatively rapidly accomplished. This advantage may be conveniently exploited without running the danger of interfering with the results, since in the subsequent stripping stage, as will be illustrated in the examples below, the characteristic voltage peaks assigned to mercury and gold are easily distinguishable.

Other parameters of interest are the size of the stirrer bar provided within the cell, which is preferably about ⅔ by length of the diameter of the cell, and the stirring rate, which is in the range of 500 to 1000 rpm, and more preferably about 950 rpm.

Having completed the deposition of the metals which may be potentially present in the sample solution onto the surface of the working electrode, said working electrode is being placed at anodic mode and the voltage applied thereto is gradually raised, preferably at a rate between 50 and 200 mV/s. The characteristic peak, which indicates that gold has been anodically stripped, is expected to be at about +0.8 to +1.0V. Upon time integrating the area of said current peak the charge required to oxidize the gold is obtained, from which the quantity of gold that was present in the tested sample may be readily obtained. A commercial software that may be used is available from Headstart Software, Ruabon, England.

The present invention provides a detection technique for the quick, simple and relatively inexpensive evaluation of the integrity of membrane systems. In the detection scheme of the invention the tested membrane system is challenged with monodispersed tracer particles, and the concentration of tracer is measured in the feed and in the permeate. Undetectable levels of the probe in the permeate indicate that the membrane skin layer is not compromised to the degree that affects virus retention.

According to one embodiment of the invention, the population of the nanoprobes contains a majority (over 51%) of the particles smaller than 3 times large than the cutoff of the membrane, and less than 0.05% of the nanoprobes particles (introduced to the feed) which are smaller than the cutoff of the membrane.

In another aspect, the present invention provides a method for the determining of the amount of pathogen (including viral) leakage through a water treatment membrane by passing a liquid that contains a substantially mono-dispersed population of nano-probes through said membrane to form a permeate and testing said permeate for the presence of said nano-probes. The viral removal percent can be estimated from the concentration of probe particles in the permeate.

Preferably, the population of the nanoprobes contains a majority (over 51%) of the particles smaller than 3 times the size of the virus that should be removed by the membrane treatment and less than 0.01% of the probe particles are smaller than the size of the virus or pathogen that should be removed by the water treatment.

According to one embodiment of the invention, the mono-dispersed biological nano-probes (e.g., fluorescent-dye-labeled MS2 bacteriophages having diameter of 28 nm) may be used in combination with the gold nano-particles (e.g., the citrate-stabilized or thiol-stabilized gold nanoparticles having an average diameter of between 10 to 15 nm) to afford a comprehensive integrity test for a virus-removal membrane.

In addition to the detection of changes in the integrity of ultrafiltration membranes in water treatment plants, the integrity test provided by the present invention may also be used by membrane manufacturers for tailoring a suitable and safe cleaning protocol (namely, cleaning agents and concentration thereof) for their membranes. As will be shown in the examples below, in tests carried out using these probes breaches in membrane were detected as early as the first appearance of small holes with an average diameter of 30 nm. It should be noted that the use of the sensitive probes of the invention enables the detection of an early breach at C•t (concentration of cleaning solution×contact time) levels of 5 g/L-h following chemically treating the membrane with cleaning (oxidizing) agents. This C•t level is three times lower than the breach detection accomplished by the conventional bubble point method, which detected breaches only after application of an oxidizing agent at a C•t level of 18 g/L-h. In order to detect a breach at such a C•t level (5 g/L-h) with the bubble point method, it will require the application of additional pressure of 10 bars, which is four times greater than the maximum pressure permitted by membrane manufacturers.

### BRIEF DESCRIPTION OF THE DRAWINGS

In the drawings:

FIG. 1 schematically illustrates a preferred electrochemical cell device for determining the presence of gold in the permeate collected from a tested membrane;

FIG. 2 schematically illustrates a set-up used for the filtration experiments.

FIG. 3 is a calibration curve for nano-probes (T4 bacteriophages) used in the present invention.

FIG. 4 is a calibration curve for nano-probes (HRP-conjugated bacteriophages) used in the present invention.

FIG. 5 is a calibration curve for nano-probes (T4 bacteriophages) used in the present invention.

FIG. 6 is a graph showing the amount of gold measured in the permeate recovered from different membranes following chemical treatment of said membranes.

FIG. 7 is a graph showing the amount of gold measured in the permeate recovered by a certain membrane following chemically treating said membrane.

FIG. 8 is a graph showing the amount of gold measured in the permeate recovered by a certain membrane following chemically treating said membrane.

FIG. 9 is a graph showing the amount of MS2 bacteriophages measured in the permeate recovered by several membranes following chemically treating said membranes.

### DETAILED DESCRIPTION OF THE INVENTION

### Examples

### Materials

### General

Deionized (DI) water (electrical resistivity>18 MΩcm) was obtained from a MilliQ water purification system. Where required, the pH of the phosphate buffer solution consisting of a 0.1 M solution of Na₂HPO₄ and KH₂PO₄ was adjusted with 0.01 M NaOH (Frutarom, Israel) or 0.01 M HCl (J. T. Baker Chemical Co., Philipsburg, N.J., USA). The exact pH values of the buffer solutions were determined with an Accumet AB15 pH meter. All glassware used was treated with piranha solution (3:7 hydrogen peroxide/concentrated sulfuric acid).

### The Tested Membranes

Seven types of UF flat-sheet membranes of typical material and size range for UF water treatment applications were used in tested in accordance with the method of the invention: cellulose acetate (CA-0.5, CA-5, CA-10), cellulose ester (CE-20), polyvinylidene fluoride (PVDF-55) and polyethersulfone (PES-15, PES-20). All the membranes were commercially available integrally skinned asymmetric membranes that differ in terms of their chemical and mechanical stability and of their resistance to organic fouling. PVDF and PES membranes are usually considered more stable but less resistant. The following table summarizes the properties of the tested membranes:

TABLE 3
Properties of tested membranes.

| | | | Average | Contact | |
|---|---|---|---|---|---|
| | | MWCO | pore size, | angle θ, ° | Permeability, |
| Type | Manufacturer | (kDa) | nm | in water | L/m² · h · bar |
| CA-0.5 | Spectrum¹ | 0.5 | 1.5 | 95 ± 1 | 12 ± 2 |
| CA-5 | Spectrum | 5 | 4 | 92 ± 1 | 16 ± 3 |
| CA-10 | Spectrum | 10 | 5 | 90 ± 1 | 19 ± 3 |
| CE-20 | Spectrum | 20 | 9 | 88 ± 1 | 44 ± 5 |
| PES-15 | FuMA-Tech² | 15 | 1 | 51 ± 1 | 90 ± 2 |
| PES-20 | Sterlitech³ | 20 | 9 | 52 ± 1 | 50 ± 3 |
| PVDF-55 | FuMA-Tech | 100 | 55 | 106 ± 1 | 250 ± 7 |

¹Spectrum Laboratories, Inc. (Rancho Dominguez, CA, USA);
²FuMA-Tech GmbH (St. Ingbert, Germany);
³Sterlitech Corporation (Kent, WA, USA)

Contact angle (as an index of membrane hydrophobicity) was determined by the sessile drop method. In this test, a ˜20-μl drop of DI water was placed onto the dried membrane surface with a microsyringe, and the air/water-surface contact angle was measured with a goniometer (Rame-Hart, Calif.) within 10 s. Contact angle measurements were performed in triplicate using separate pieces of membrane. Contact angles greater than 90° indicate that the membrane is hydrophobic, and extra pressure should be applied to push the water through the membrane. In this example, only the PVDF-55 was found to be hydrophobic. Polyether sulfone membranes were, on average, more hydrophilic than those made of cellulose acetate.

Membrane permeability to pure water was obtained by filtration of DI water through the membrane in test cell 22 for 30 min at 1 bar N₂ pressure after the membranes had been soaked in DI water at 30° C. for 1 h to remove glycerin.

### Filtration Protocol

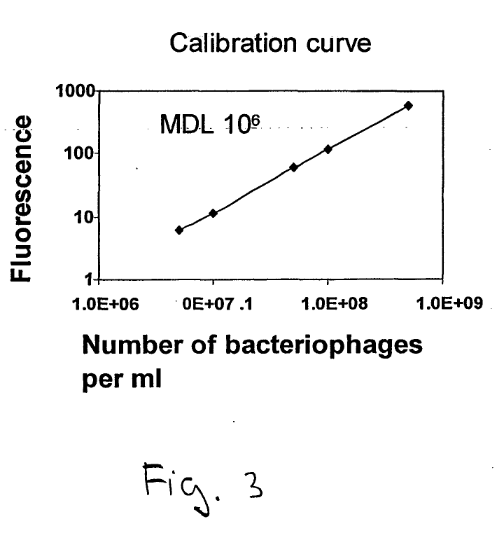

The laboratory-scale 500-ml stirred cell (Spectrum Inc., Ltd.) which was used for passing the nano-probes through the membranes is illustrated in FIG. 2.

The cell 22 consisted of an acrylic glass cylinder 21 capable of withstanding an internal pressure equal to, or smaller than 5 bars (≦5). The feed solution in the acrylic glass cylinder 21 was stirred with a magnetic stirrer 23, whose stirrer 23s speed could be varied between 0 and 400 rpm. The feed opening is indicated by numeral 20. The cell 22 was fitted with a back-pressure controller (not shown) for control of transmembrane pressure and provisions for 76-mm diameter membranes. Circular pieces of the membranes 25 to be tested, approximately 150 nm thick, were supported on a Teflon base. The entire system was mounted on a stainless steel frame. The feed solution was pressurized by nitrogen 28 at a constant pressure of 2 atm throughout the testing of the various membranes 25. Pressure was set by a pressure regulator 27 at a desired set-point. Numerals 24 and 26 indicate means for the collection of the permeate generated by the membrane.

### Example 1

### Testing the Integrity of Intact Membranes by Means of MS2 Bacteriophages

### Preparation

The initial stock of MS2 bacteriophages (DSM-No 13767) along with Escherichia coli host cells (DSM-No 5695) were purchased from German Collection of Microorganisms and Cell Cultures (DSMZ, Braunschweig, Germany). The bacteriophages were prepared by inoculation of 1:1 ratio of phages and host cells to the overlayer. After 24 hours incubation at 37° C., the overlayer containing the infected bacterial cells was scrapped into 50-ml tubes. Purification of the bacteriophage culture was accomplished by chloroform extraction. The stock was enumerated by plaque-assay method using the double-layer technique.

The initial titer was diluted to the concentration of 2×10¹¹ PFU/ml and labeled with fluorescein-5-isothiocyanate, FITC, fluorescein, 5-(4,6-dichlorotriazinyl)aminofluorescein, 5-DTAF, or rhodamine B. For FITC and DTAF, 1.2 mL of MS2 phages in 0.1 M borate buffer, pH 9.2, were mixed with 0.021 g FITC or 0.0102 g 5-DTAF and 5 mL N,N-Dymethylformamide (DMF). The solutions were stirred overnight at 4° C. and then purified by dialysis. Rhodamine B and fluorescein labeling of MS2 phages were performed by mixing 0.2 g of DEC, 1-[3-(Dimethylamino) propyl]-3-ethylcarbodiimide hydrochloride (Aldrich) coupling agent and 0.02 g of the dye (Rhodamine B (Aldrich) or sodium fluorescein (BDH)) in 25 ml of bacteriophage stock solution in phosphate buffer (pH 5.6). The labeled bacteriophage mixture was purified by membrane dialysis (The Scientific Instrument Center Ltd., London, UK) under stirring to remove low molecular weight organic compounds and free dye molecules. Approximately three days of dialysis were needed to obtain leak free fluorescein and Rhodamine fluorescent-labeled bacteriophages, and several hours (ca 10) dialysis were sufficient to achieve leak free FITC and DTAF labeled phages. The modified bacteriophages were stored at 4° C. in the dark. No changes in fluorescence of the labeled bacteriophages were detected during two months storage in a refrigerator. The average size of the labeled bacteriophages was 30+/−10 nm by SEM studies.

### The Test

One ml of the obtained stock was withdrawn from the storage, mixed with 50 ml of DI water and filtered through the membrane according to the filtration protocol described above. Ten ml of the permeate were collected and analyzed with Perkin-Elmer LS-50B FL fluorescence spectrometer (Perkin Elmer, Norwalk, USA) equipped with a 1-cm optical path length cuvette. The obtained intensity of the sample was compared to the calibration curve obtained by fluorescence measurements of suspension with known concentration of died MS2. In case no signal was obtained the membrane was deemed to be intact. The intensity of signal, if obtained, was transferred into the concentration of probe in the permeate.

### Results

The bacteriophages were undetectable in the permeate of all membranes except PVDF-55. For the later, 8×109 PFU/ml of the viruses were counted in the permeate.

### Example 2

### Testing the Integrity of Intact Membranes by Means of T4 Bacteriophages

### 2A: Fluorescent Dye Labeling of T4 Bacteriophages

### Preparation

Five ml of an initial solution containing 10¹⁰ (ten to the power of ten) T4 phages were washed by introducing them into a dialysis membrane bag, and then they were left overnight in one liter of 10 mM of HEPES buffer at 4° C., to give Solution A. Into 5 ml of Solution A were added 25 mg of commercial Sulfo-N-hydroxysuccinimidobiotine (EZ-link Sulfo-NHS-Biotin of Pierce, Rockford, Ill. USA) and the mixture was left to react for 24 hours at 4° C., to give Solution B.

250 microliter of solution B were mixed with 500 microliter of streptavidin-fluorescein conjugate (commercially available, from Amersham Biosciences UK limited, Amersham Place, Little Chalfont Buckinghamshire, England. Catalogue number RPN1232-2ML) and the resulting solution was left to react for 24 hours at 4° C. The obtained solution was washed for two days by introducing it into a dialysis membrane bag and immersing it in 1 liter of 10 mM HEPES solution. The washing solution was replaced 7 times during this time interval.

The calibration curve for the detection of fluorescein labeled phages was obtained by means of fluorescence detection, and the results are shown in FIG. 3, which demonstrates a calibration curve showing the dependence of the fluorescence intensity on the concentration of T4 phages. The minimum detection limit was 1 million phages/ml and linear signal—concentration dependence was demonstrated.

### 2B: Enzymatic Labeling of T4 Bacteriophages

### Preparation

250 microliter of solution B (see details of preparation in Example 2A) were mixed with 500 microliter of streptavidin-HRP conjugate (commercially available as catalogue number RPN1231-2ML from Amersham Biosciences UK limited, Amersham Place, Little Chalfont Buckinghamshire, England) and the resulting solution was left to react for 24 hours at 4° C. The resulting solution was subsequently washed for two days by introducing it into a dialysis membrane bag and immersing it in 1 liter of 10 mM HEPES solution. The washing solution was replaced 7 times during this time interval. The thus obtained solution served as a stock HRP-bacteriophage conjugate solution in our further studies.

We determined the calibration curve for the detection of HRP labeled phages by two methods:

### 2.B.1. Detection by Biocatalytic Oxidation of Pyrogallol

We prepared the following solutions:

A1: 100 mM potassium phosphate buffer at pH 6

A2: 0.5% (w/w) hydrogen peroxide solution.

A3: 5% (w/v) of pyrogallol solution

To 3 ml of deionized water we added 280 microliter solution A1 and then 240 microliter of solution A2 and 480 microliter of solution A3 and then introduced 5 microliter of a solution containing diluted stock HRP-T4 conjugate solution and the left the solution to react for 30 minutes. Then we measured the absorbance at a wavelength of 420 nm by Cary 1 Spectrophotometer (of Varian Ltd.).

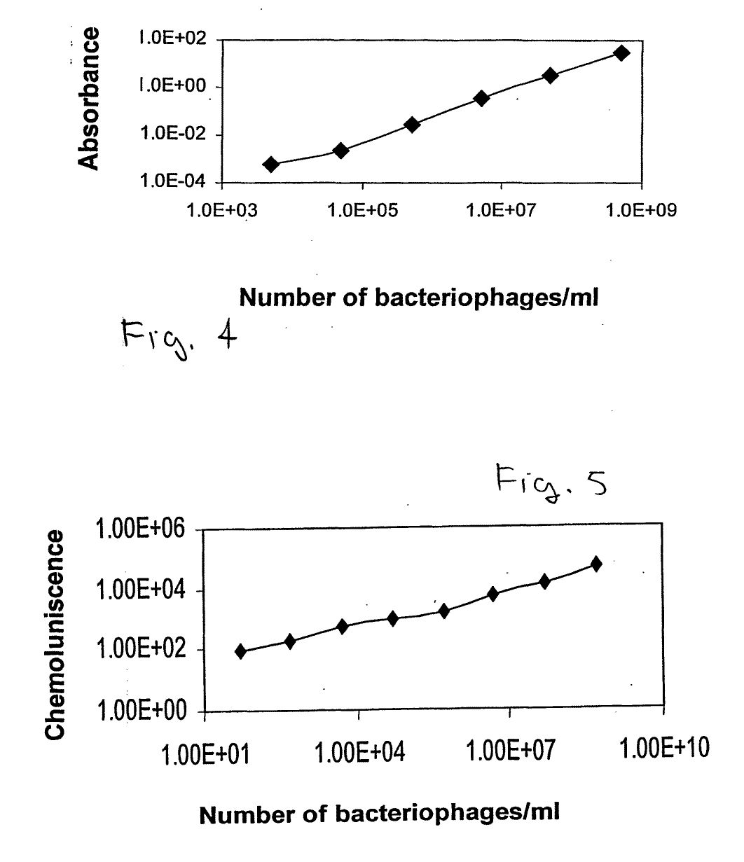

FIG. 4 demonstrates the calibration curve obtained by this method. The detection limit by this method is approximately 1000 phages per ml which is over two orders of magnitude better than fluorescein conjugated bacteriophages. The superior sensitivity obtained by this method is caused by the catalytic action of the HRP. Each HRP catalyze the conversion of many pyrogallol dye molecules and the purpurogallin oxidation product accumulates and gives highly colored solution.

2.B.2 Detection by Enhanced Chemiluminescence (ECL) method.
We prepared the following solutions:

Solution B1: 10 mg of p-iodophenol was introduced to 1 ml DMSO.

Solution B2: 10 mg luminol was introduced to 1 mL of DMSO.

Solution B3: 730 microliter of solution B1 and 950 microliter of solution B2 were added to 10 ml of 0.1 M tris buffer, pH 8.5

The measurement was conducted by introducing into a microtiter well 200 microliters of solution A2, 100 microliter of solution B3 and 20 microliter of diluted HRP-T4 conjugated stock solution. We measured the luminescence response after 1 minute.

FIG. 5 demonstrates the calibration curve obtained for T4-HRP by enhanced chemical luminescence method. The minimum detection limit is further reduced and the sensitivity is enhanced compared to previous methods of detection.

Permeability results: the bacteriophages were undetectable in the permeate of CA-5 membrane even after introduction of 10⁹ phages per 1 into the membrane system, showing (with the current detection level) over 5 logs of virus removal.

### Example 3

### Testing the Integrity of Intact Membranes by Means of Proteins

### Preparation

Bovine serum albumin (cold alcohol precipitated BSA) was purchased from Sigma-Aldrich. Israel Ltd. (Rehovot, Israel). A 0.15-g sample was dissolved in 500 ml deionized water (RO quality) to form a 0.3 g/L protein solution. The solution was labeled with fluorescein-5-isothiocyanate, FITC, fluorescein, 5-(4,6-dichlorotriazinyl)aminofluorescein, 5-DTAF, or rhodamine B. Same procedure as in working Example 1 was used. The labeled protein mixture was purified by membrane dialysis (The Scientific Instrument Center Ltd., London, UK) under stirring.

### The Test

Fifty ml of the obtained stock was withdrew from the storage and filtered through the membrane. Ten ml of the permeate were collected and analyzed with Perkin-Elmer LS-50B FL fluorescence spectrometer (Perkin Elmer, Norwalk, USA) equipped with a 1-cm optical path length cuvette. The obtained intensity of the sample was compared to the calibration curve obtained by fluorescence measurements of suspension with known concentration of died BSA.

The Stokes radius of BSA was determined from its diffusivity in a solution by using the following Stokes-Einstein equation

$${D}_{AB} = \frac{kT}{6 π r μ} , \qquad ( 1 )$$

Where D_{AB}=diffusivity of BSA in the water, k=Boltzmann constant; T=the absolute temperature; r=the solute particle radii; μ=water viscosity. The diffusivity can also be approximated by using the semiempirical equation of Polson, which is recommended for molecular weights above 1000 Da

$${D}_{AB} = \frac{9.4 \cdot {10}^{- 15} T}{{μ ( {M}_{A} )}^{1 / 3}} \qquad ( 2 )$$

where M_{A} is the molecular weight of the large molecule in Da. Combining the Equations (1) and (2), the approximate radii of BSA (67 kDa) as a particle is

r (nm)=0.078M^{0.33}=3.05 nm (3)

In case no signal was obtained the membrane was deemed to be intact. The intensity of signal, if obtained, was transferred into the concentration of the probe in the permeate. Compared to the measured concentration of BSA in the feed, the presence of dye labeled BSA in the permeate indicates that the membrane is damaged to the level to contain holes of 6.1 nm and higher.

### Example 4

### Testing the Integrity of Intact Membranes by Means of Gold-Nanoparticles

### Preparation of Citrate-Stabilized Gold Nanoparticles

A colloidal aqueous suspension of citrate-stabilized gold nanoparticles was prepared by adding 4 ml of a 1% solution of trisodium citrate to 40 ml of a 0.01% (w/v) solution of HAuCl₄ according to the procedure described by G. Frens in Controlled nucleation for the regulation of particle size in monodisperse gold solutions, Nature Phys. Sci. 241 (1973), 20-22.] The mixture was stirred for 5 min under gentle boiling, cooled to room temperature, and stored at 4° C. The obtained colloids were composed of an internal core of gold metal coated with a layer of weakly bound acetone dicarboxylate and other citrate oxidation products (A. N. Shipway, E. Katz, I. Willner, Nanoparticle arrays on surfaces for electronic, optical, and sensor applications, Chem. Phys. Chem. 1 (2000), 18-52.) The colloids possessed a negative surface charge in water as a result of partial ionization of the carboxylic groups, starting around pKa 3.1.

### Preparation of Thiol-Stabilized Gold Nanoparticles

Another set of nanoparticles were stereo-stabilized with mercaptopropionate ions (thiol-stabilization) known for their ability to form strong Au—S bonds (M. Brust, J. Fink, D. Bethell, D. J. Schiffrin, C. Kiely, Synthesis and reactions of functionalized gold nanoparticles. J. Chem. Soc. Chem. Comm. 16 (1995), 1655-1656; K. Aslan, V. H. Perez-Luna, Surface modification of colloidal gold by chemisorption of alkanethiols in the presence of a nonionic surfactant. Langmuir 18 (2002), 6059-6065). A colloidal suspension of thiol-stabilized gold nanoparticles was prepared as follows. The citrate-stabilized nanoparticle solution described in Preparation 1 was stirred overnight with a solution of 0.01 mM 11 mercaptoundecanoic acid, resulting in the formation of a stable particle solution. The solution was dialyzed against DI water for three days to remove citrate and thiol debris.

Examination of the UV-vis spectrum of the citrate-stabilized solution that had been left to stand for two months showed it to be relatively stable, since only one peak (at 535 nm) was evident: a shift in the peak or the appearance of a second peak would have indicated decomposition or precipitation.

### The Test

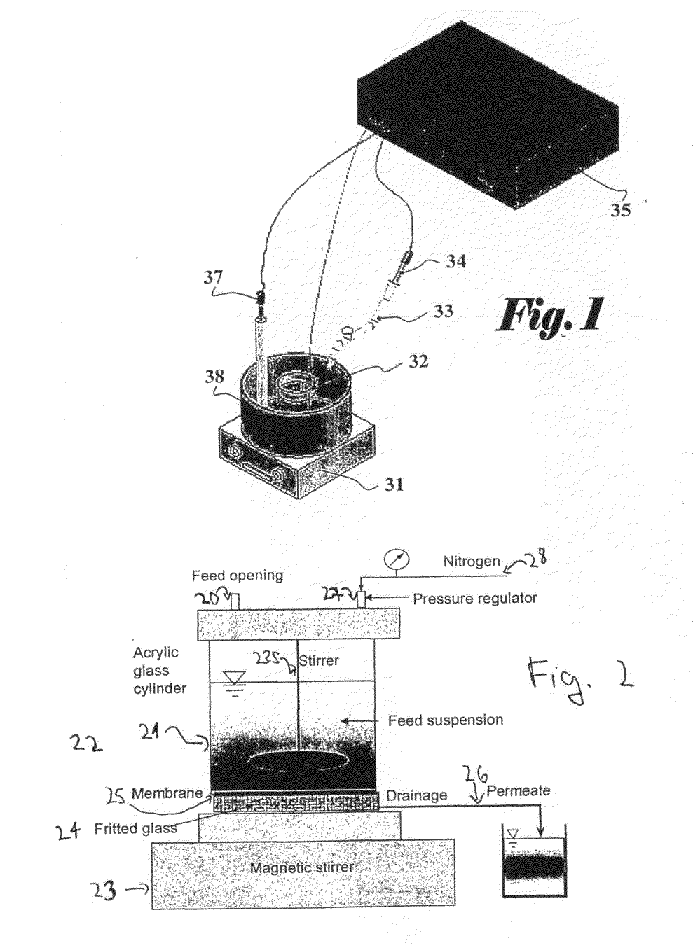

A colloidal aqueous suspension of the citrate-stabilized gold prepared as described above was withdrawn from the cool room, diluted 10 times to obtain 5.2 ppm initial gold solution and passed through the tested membrane according to the filtration protocol described hereinabove. Five ml of permeate were collected and mixed with concentrated aqua regia (3 M HCl:1 M HNO3) under continuous stirring (950 rpm) performed with magnetic stirrer. The stirring was performed inside a 20-ml acrylic cell that was later used for gold determination according to the set-up illustrated in FIG. 1. The stirring was performed for 2 minutes and at the same time 100 ppm mercury as mercury (II) chloride was added. The sample was held at −0.7 V for 30 s followed by 10 cycles each of −0.6 V for 10 s followed by +0.3 V for 1 s. Stripping was performed by six cycles of linear potential scan from −0.7 V to 1.0 V at a scanning rate of 100 mV/s. All measurements were performed with a commercially available 263 A potentiostat-galvanostat (EG&G Princeton Applied Research, NJ, USA). After the stripping step, the cell was rinsed twice with DI water and twice with dilute aqua regia (0.1 M HCl/0.4 M HNO₃ in DI water), at least 10 s per wash. After rinsing, no residual peaks were detected within the potential range, and the electrodes could be used for subsequent measurements. The charge required to strip the deposited gold was given by time integration of the current peak (HeadStart Software, Ruabon, England).

### Results

The gold nanoparticles were undetectable in the permeate of all membranes except PVDF-55.

### Example 5

### Testing the Integrity of Intact Membranes by Means of DNA Plasmids

### Preparation

Two types of double stranded circular DNA plasmids were used: a 4.5 kilo base pair (kb) pGEMR and a 9.5 kb pHE4-ADR. A NucleoBond PC 500 isolation kit (Macherey-Nagel, Düren, Germany) was used for isolation of plasmids. The plasmids were diluted in DI water to form the initial suspension of 0.33 μg/ml in 100 ml. Only 50 ml of feed suspension was transferred through membranes at constant temperature of 20±1° C. and at pH 6.0.

A 439 bp fragment of pHE4-ADR plasmid (9.5 kb), before and after filtration through the membrane, was amplified using Un4(d) GCATATGATGTAGCGAAACAAGCC and Un4(r) GCGTGACATACCCATTTCCAGGTCC primers, with a Mastercycler gradient thermocycler (Eppendorf, Westbury, N.Y.). Reaction mixtures included a 12.5 μl ReddyMix (PCR Master mix containing 1.5 mM MgCl₂ and 0.2 mM concentration of each deoxynucleoside triphosphate) (ABgene, Surrey, UK), 1 pmol of each of the forward and reverse primers, 1 to 2 μl of the sample preparation, plus water to bring the total volume to 25 μl. An initial denaturation-hot start of 2 min at 94° C. was followed by 30 cycles of the following incubation pattern: 94° C. for 30 sec, 54° C. for 30 sec, and 72° C. for 45 sec. PCR products were purified by electrophoresis through a 0.8% agarose gel (Sigma), stained with ethidium bromide and visualized on a UV transilluminator.

Concentration of plasmid DNA in the feed solution and concentration of plasmid DNA in the permeate fraction were determined by the real-time PCR method. Real-time PCR analysis was performed using the following sets of primers: 341F CCTACGGGAGGCAGCAG and 518R ATTACCGCGGCTGCTGG, Un4(d) GCATATGATGTAGCGAAACAAGCC and Un4-r(2) CTCAGCGTACTGAATTTGAGCG, Un4-d(2) GCGTATCTCAAAATGTCCATCTCC and Un4(r) GCGTGACATACCCATTTCCAGGTCC. Quantification of bacterial DNA was performed in the ABI prism 7000 Sequence Detection System (Applied Biosystems) using Absolute QPCR SYBR Green ROX Mix (ABgene, Surrey, UK) on a 96-well optical plate. The PCR reaction consisted of 10 μl of Absolute QPCR SYBR Green ROX Mix, 150 nM each of forward and reverse primers, and 5.0 μl of each DNA template, in a total volume of 20 μl. The thermal cycling conditions were as follows: 2 min at 50° C., 15 min at 95° C., followed by 40 rounds of 15 sec at 95° C. and 1 min at 60° C. To verify that the used primer pair produced only a single specific product, a dissociation protocol was added after thermocycling, to determine dissociation of the PCR products from 60° C. to 95° C.

The pGEMR (4.5 kb) and pHE4-ADR (9.5 kb) plasmids were used for detection of membrane rejection ability and as standards for the calibration curves for quantification at six serial dilution points (in 10-fold steps). All runs included a no-template control. Reproducibility of SYBR Green real-time PCR was assessed by running samples independently on different days. The PCR product was verified with ethidium bromide-stained 2% agarose gels (SeaKemR LE Agarose; FMC BioProducts, Rockland, Me.).

The ABI prism 7000 Sequence Detection System and SDS Software were used for data analysis. The ABI prism 7000 monitors the fluorescence resonance energy transfer (with a SYBR Green fluorophore) of reaction mixtures, just before the denaturizing step of each amplification cycle and records the cycle number at which fluorescence crosses a specific threshold cycle (Ct) value. The cycle number at which the signal is first detected is correlated with the original concentration of the DNA template, while the starting copy number of amplicons is inversely proportional to the real time Ct. The plasmids, both for detection of membrane rejection ability and as standards were assayed in triplicate. Standard curves were obtained by plotting the Ct value of each 10-fold dilution series of plasmids.

The size of plasmid DNA in solution was measured by atomic force microscopy (AFM) and dynamic light scattering (DLS). For AFM, purified plasmid DNA samples were suspended in 1 mM NiCl₂—10 mM Hepes (final concentration of DNA was 1 ng/μl), 20 μl dropped onto freshly cleaved mica, and incubated for 5 min at room temperature. After rinsing with 1 ml DI water the samples were dried. AFM measurements were performed at ambient conditions using a Digital Instrument Dimension 3100 (Digital Instruments, Santa-Barbara, Calif.) mounted on an active anti-vibration table. A 100 μm scanner was used. The 512×512 pixel images were taken in tapping mode with a scan size of up to 5 μm at a scan rate of 1 Hz. The hydrodynamic radius of the plasmid DNA was determined with a CGS-3 goniometer (ALV, Langen, Germany) equipped with an He—Ne 22 mW 632.8 nm laser. The spectra were collected at angles varying from 30° to 150°. The diffusion coefficient was determined at 30°. The autocorrelation function was calculated by using an ALV/LSE 5003 multiple tau digital correlator (ALV, Langen, Germany). Before each measurement, the sample was passed through a 0.8 μm filter (CA membrane) to obtain the lowest noise possible. Approximately 1 ml of the sample was inserted into a glass vial sealed with Teflon paper. Each measurement was performed at 20° C. on the basis of 20 runs of 10 seconds; runs with a high baseline level were disregarded.

The absolute size of DNA molecule measured by AFM and DLS, as well as calculated using the Stokes-Einstein equation (see working example 3), is 350 nm. In case no signal was obtained the membrane was deemed to be intact. The intensity of signal, if obtained, was transferred into the concentration of the probe in the permeate. Compared to the measured concentration of BSA in the feed, the presence of died BSA in the permeate indicates that the membrane is damaged to the level to contain holes of 350 nm and higher.

### Example 6

### Testing the Integrity of Chemically-Treated Membranes by Means of Nano-Probes

The membranes identified in Table 3 above were chemically cleaned. The cleaning of the membranes was performed with commercially available bleach NaOCl (18 g/L free chlorine) at concentrations of 20, 40, 70 and 400 mg/L or with 0.3% NaOH (Frutarom, Israel). Introduction of NaOH raised the pH to 11. Oxidation with hypochlorite was performed at pH 6.8. In some tests, the pH was adjusted with dilute HCl (J. T. Baker Chemical Co., Philipsburg, N.J.) or NaOH to the desirable value. Concentration of free chlorine in the soaking solution was determined by the 4500-Cl B iodometric method I (Standard Methods for Examination of Water and Wastewater, 20^{th} edition by L. S. Clescerl, A. E. Greenberg, A. D. Eaton, American Public Health Association, 1999) using the following relationship:

$$mg Cl as {Cl}_{2} / ml = \frac{A \cdot 0.01 \cdot 35.45}{ml sample} \qquad ( 1 )$$

where A is the titrating solution milliliters, 0.01 is the normality of Na₂S₂O₃, 35.45 is the molecular weight of chlorine. Titration of the blank showed that there was no free chlorine in the deionized water.

The chemically treated membranes were subjected to the tests described in the previous examples (using either gold nanoparticles or fluorescein-5-isothiocyanante (FITC)-labeled MS2 bacteriophages) and the results will now be described with reference to the accompanying Figures.

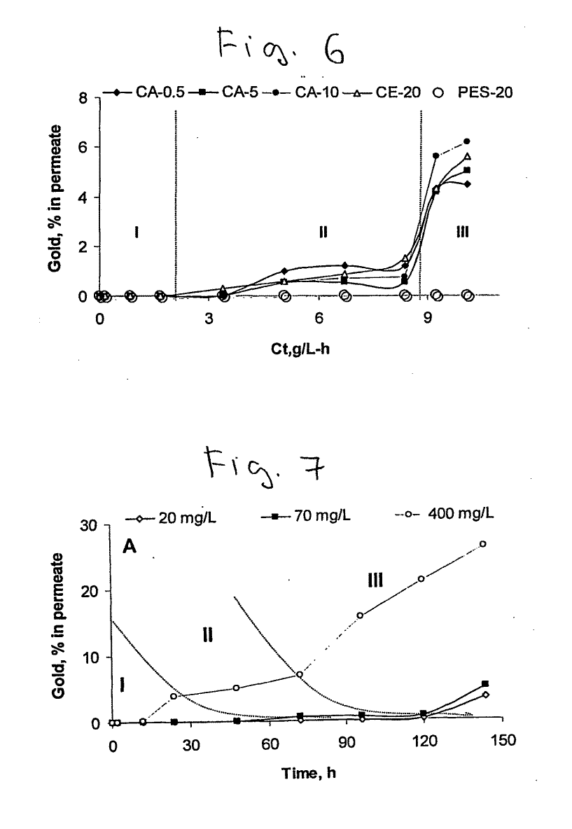

FIG. 6 presents the first set of experiments performed on polyethersulfone membrane PES-20 and on cellulose membranes CA-0.5, CA-5, CA-10 and CE-20. No detectable levels of gold were observed for the untreated membranes and for the membranes that had been soaked in the 70 mg/L free chlorine solution for 1, 2, 4, 12, or 24 h. The solution pH had been kept at constant at 7.3-7.4, ensuring equal concentrations of HOCl and OCl⁻ ions. After 48 h of treatment, the CE-20 membrane exhibited approximately 0.1% leakage of the gold probe solution into the permeate. At C•t levels of 5 g/L˜h, all four cellulose membranes showed some degree of disintegration, i.e., 0.55 to 1% leakage. The trend continued for all four types of cellulose membranes for cleaning intervals of 72-120 h, with the gold concentration varying between 0.6 and 1.5%. That level corresponds to 5 g/L-h C•t value. After 120 h of oxidation, corresponding to a C•t value of 10 g/L-h, the concentration of gold in the permeate increased to 4-6% of the initial probe. This massive leakage was linked to the first signs of membrane disintegration depicted in FIG. 7C for 18 g/L-h C•t contact value. In FIG. 6, the areas of undetectable gold levels, a plateau of small concentrations in the permeate, and the complete disintegration of the membrane were designated as areas I, II and III, respectively. No signs of disintegration of the polyethersulfone membrane PES-20 were depicted. The experiments with PES-20 membrane were continued up to C•t values of 50 g/L-h without any detectable concentrations of gold in the permeate.

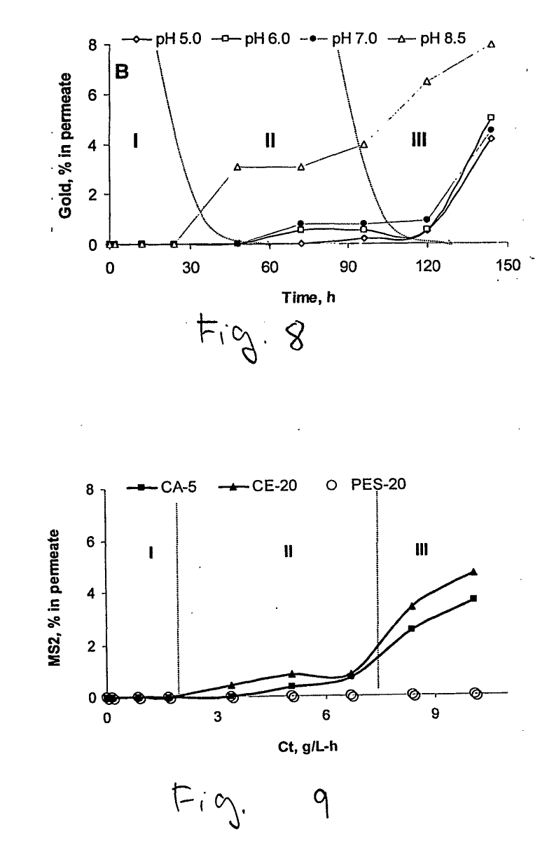

FIGS. 7 and 8 presents the results of studies of the kinetics of disintegration for CE-20 membranes at different concentrations of NaOCl (FIG. 7) and different pH levels (FIG. 8). Application of 20 and 70 mg/L free chlorine resulted in virtually the same level of gold transition. Higher concentrations of free chlorine, such as 400 mg/L (FIG. 7), caused faster destruction of the membrane skin layer and earlier appearance of gold nanoparticles in the permeate. Experiments performed at pH 5, 6, and 7 gave almost similar results, but the pattern of deterioration was completely different for the pH 8.5 treatment (FIG. 8). At this pH, the percent of gold nanoparticles in the permeate was consistently higher, as can be seen from FIG. 8. The observed phenomenon can be attributed to the low chemical stability of cellulose membranes at this pH value, as was already mentioned above.

A similar trend in membrane deterioration upon contact with aqueous chlorine was also observed for CA-5 and CE-20 membranes when fluorescent-dyed MS2 bacteriophages were used as the integrity probes (FIG. 9). The soaking solution contained 70 mg/L free chlorine. Both CA-5 and CE-20 membranes were found to be chemically unstable under free chlorine attack and exhibited some degree of penetration of MS2 bacteriophages into the permeate. Penetration of the bacteriophages into the permeate was first detected for the CE-20 membrane after 48 h of NaOCl soaking. After 72 h, both CA-5 and CE-20 exhibited some degree of disintegration. Once again, the third period of disintegration was observed at C•t values of 10 g/L-h. The similar experiments performed with PES-20 membrane did not result in any detectable concentration of MS-2 bacteriophages in the permeate.

1. A method for evaluating the integrity of microfiltration, ultrafiltration and nanofiltration membranes, which method comprises passing a liquid that contains a substantially mono-dispersed population of nano-probes through said membrane to form a permeate and testing said permeate for the presence of said nano-probes, wherein the non-detection of said nano-probes in said permeate indicates that said membrane is substantially intact.

2. The method according to claim 1, wherein the filtration membrane is a virus-retaining membrane.

3. A method according to claim 1, wherein the nano-probes are biological entities selected from the group consisting of viruses, bacteriophages, proteins and nucleic acids, wherein said biological entities are coupled to a reporting molecule, wherein said reporting molecule is either an optically or electrochemically detectable compound, or said reporting molecule is capable participating in a chemical transformation or a physical process which involve the formation of one or more intermediates and/or products that are optically or electrochemically detectable.

4. The method according to claim 3, wherein the nano-probes are viruses or bacteriophages that are labeled with a fluorescent dye.

5. A method according to claim 4, wherein the nano-probe is MS2 bacteriophage labeled with a fluorescent dye.

6. A method according to claim 3, wherein the reporting molecule that is coupled to the nano-probe is a catalyst which catalyzes a chemical transformation of a starting material into a product that is optically detectable, and wherein the testing of the permeate for the presence of the nano-probe includes performing said reaction that is catalyzed by said catalyst and optically determining the presence of said product.

7. A method according to claim 6, wherein the nano-probe is a bacteriophage conjugated to an enzyme that is capable of catalyzing a reaction that produces two or more optically or electrochemically detectable products per one molecule of starting material.

8. A method according to claim 7, wherein the enzyme is a peroxidase or glucose oxidase enzyme.

9. A method according to claim 8, wherein the nano-probe is T4 bacteriophage that is conjugated to the enzyme horse radish peroxidase.

10. A method according to claim 1 that is carried out on-line, wherein the liquid that contains the nano-probes is added to the feed of a membrane module and one or more sensors are used to detect said nano-probes in the permeate by measuring one or more physical properties associated with said nano-probe.

11. A method for evaluating the integrity of a water treatment membrane which method comprises passing gold nanoparticles dispersed in an aqueous medium through said membrane to form a permeate, sampling the permeate, and electrochemically testing the sample for the presence of gold, wherein the non-detection of said gold in said permeate indicates that said membrane is substantially intact and/or is suitable for intended purpose.

12. A method according to claim 11, wherein the electrochemical testing includes introducing an oxidizing agent into the sample to form gold ions in a solution, depositing metal ions present in the solution onto a working electrode and subsequently applying a progressively varied anodic potential to said working electrode capable of releasing gold therefrom, whereby the gold quantity is determined.

13. A method for the determining of the amount of pathogen (including viral) leakage through a water treatment membrane by passing a liquid that contains a substantially mono-dispersed population of nano-probes through said membrane to form a permeate and testing said permeate for the presence of said nano-probes.

14. Bacteriophage conjugated to a catalytic entity that is capable of catalyzing a reaction that produces two or more optically or electrochemically detectable products per one molecule of substrate.

15. Inactivated virus conjugated to a catalytic entity, wherein said catalytic entity is capable of catalyzing a reaction that produces two or more optically or electrochemically detectable products per one molecule of substrate.

16. A virus conjugated to an enzyme or DNAZYME entity, wherein said entity is capable of catalyzing a reaction that produces two or more optically or electrochemically detectable products per one molecule of substrate.

17. A bacteriophage according to claim 14, wherein the catalytic entity which is appended to the bacteriophage changes the size of the virus by not more than 25% relative to its original size, and wherein the sign of the zeta potential of the original phage is not altered by the conjugation of said catalytic entity.

18. A bacteriophage according to claim 17, wherein the catalytic entity conjugated thereto is a peroxidase or glucose oxidase enzyme.
